(19) United States
(12) Patent Application Publication
(10) Pub. No.: US 2006/0259014 A1
(43) Pub. Date: Nov. 16, 2006

(54) Aspirator sleeve and suction handle

(75) Inventor: Richard J. Yarger, Yakima, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SurgiMark, Inc., Yakima, WA (US)

(21) Appl. No.: 11/405,270

(22) Filed: Apr. 17, 2006

Related U.S. Application Data
(63) Continuation-in-part of application No. 10/969,276, filed on Oct. 19, 2004, now Pat. No. 7,066,903.
Application No. 10/969,276 is a continuation of application No. 10/153,420, filed on May 22, 2002, now abandoned.

(51) Int. Cl. A61M 27/00

(52) U.S. Cl. 604/541

(57) ABSTRACT

A sleeve and aspirator tip combination includes a surgical aspirator tip (103) couplable to a surgical aspirator sleeve (40). The surgical aspirator sleeve (40) includes spaced orifices (62) that provide communication between the external environment and the internal channel of the sleeve (40). The aspirator tip (13) includes at least one longitudinal exterior groove (74). At least one venting channel (80) is formed between the at least one longitudinal exterior groove (74) and the interior of the aspirator sleeve (40), allowing airflow between the external environment and the interior of the sleeve (40). Locking means secure the sleeve (40) to the aspirator tip (130) and prevent longitudinal and rotational movement of the sleeve (40). Alignment means guide the sleeve (40) onto the tip (130) to properly mate the sleeve (40) and aspirator tip (103), thereby ensuring formation of at least one venting channel (80) therebetween.

### CROSS-REFERENCE TO RELATED APPLICATION

This application is a Continuation-in-Part of prior U.S. patent application Ser. No. 10/969,276, filed Oct. 19, 2004, which is a continuation of application Ser. No. 10/153,420, filed May 22, 2002, the specification of which are hereby incorporated.

### TECHNICAL FIELD

This invention relates generally to surgical aspirators and surgical aspirator tip and sleeve combinations, and more particularly to surgical aspirator tip and sleeve combinations that allow ventilating air to flow into the interior of the sleeve that is independent of the inflow of gases, fluids, and materials through the small holes in the sleeve.

### BACKGROUND

Surgical aspirators are used to remove fluids from the body of the patient. A surgical aspirator typically includes a tip that is inserted into a surgical site, wound, or other bodily orifice. The tip is generally elongated in shape and may include a handle or grip section to facilitate using and holding the aspirator. The proximal end of the tip is connected to a tube that is connected to a suction pump that provides suction to the tip. The distal end of the aspirator tip is inserted into the patient and has one or more openings into which gases, fluids, and materials may flow.

Pieces of tissue and other debris may be suspended in the fluids and can clog the aspirator tip. Thus, the distal end of the aspirator tip may be covered with a sleeve that is formed with a plurality of small holes. The holes prevent the tissue from reaching the opening of the aspirator tip while allowing the fluid being evacuated to flow into the sleeve through the holes.

This action could be further enhanced by using internal projections defined on the interior surface of the sleeve to maintain the position of the sleeve relative to the aspirator tip. Projections may also be used to ensure adequate space between the aspirator tip and the sleeve. Therefore, fluids and small debris may flow freely to or through the aspirator tip end opening.

Venting channels may additionally be formed between the sleeve and tip to sustain uniform distribution of suction in the event that the holes in the sleeve become clogged. The venting channels should be properly aligned with the sleeve to ensure that airflow reaches the interior of the sleeve if any of the holes become clogged. Without such airflow, suction will no longer be uniformly distributed among the unclogged holes. This may result in excess suction in particular areas of the sleeve that may pull surrounding tissue, thereby causing injury to the patient. It would be beneficial to use a sleeve locking mechanism to secure the position of the sleeve relative to the aspirator tip such that the venting channels are maintained between the sleeve and tip during use.

Based on the foregoing, a need exists for an improved surgical aspirator tip and sleeve combination that allows air flow into the interior of the sleeve and towards the tip end opening and through properly aligned venting channels existing between the sleeve and tip.

### SUMMARY

One embodiment of a sleeve and aspirator tip combination formed in accordance with the present invention includes a surgical aspirator tip comprising an enlarged medial portion having an internal channel and an external medial portion surface, where the medial portion includes at least one longitudinal groove formed on the external medial portion surface. A hollow tubular neck member having an exterior surface extends distally from the medial portion, and the hollow tubular neck member is diametrically no larger than the enlarged medial portion. The aspirator tip also includes a tip end portion on the distal end of the hollow tubular neck member opposite the medial portion. The tip end portion defines a tip end opening and includes at least two tip end projections extending generally transversely to the hollow tubular neck member, where the tip end projections form and define tip end grooves therebetween.

The surgical aspirator sleeve includes an elongate, nominally straight tubular body having an internal sleeve surface and an external sleeve surface, where the tubular body defines an internal channel having an open aspirator sleeve end portion and an enclosed, distal tip sleeve end portion. The tubular body defines plural, spaced orifices at a spaced distance proximately from the distal tip sleeve end portion. The orifices provide communication between the external environment and internal channel of the tubular body to allow passage of liquid and small material to be aspirated into the internal channel defined by the tubular body. The tubular body may be formed from resilient, deformable material over the hollow tubular neck member of the surgical aspirator tip to receive the hollow tubular neck member of the aspirator tip therein and to assume the profile of the hollow tubular neck member of the aspirator tip. The size of the internal channel of the tubular body is sufficiently larger than the exterior of the hollow tubular neck member to enable the liquid and small material passing into the channel through the orifices to flow through the internal channel between the interior of the tubular body and the exterior of the hollow tubular neck member toward the distal tip end of the hollow tubular neck member. The aspirator sleeve also includes at least two sleeve tip end projections formed on the interior surface of the sleeve at the sleeve distal tip end portion, where the sleeve tip end projections are configured to selectively engage the tip end projections.

There is at least one venting channel in communication with the interior of the sleeve and the external environment formed between the at least one longitudinal exterior groove of the enlarged medial portion and the internal surface of the rearward aspirator sleeve end, allowing airflow between the external environment and the interior of the sleeve.

The aspirator sleeve is engageable with the aspirator tip such that the sleeve tip end projections may abut the tip end projections to form a gap between the sleeve and tip. Locking means secure the sleeve tubular body to the aspirator tip and prevent longitudinal and rotational movement therebetween. An alignment system guides the sleeve onto the tip to properly mate the tubular body and aspirator tip, thereby ensuring formation of at least one venting channel therebetween.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a side elevational view of a surgical aspirator tip and a surgical aspirator sleeve;

FIG. 2 is a side elevational view of the surgical aspirator tip of FIG. 1;

FIG. 3A is a longitudinal cross-section view of the surgical aspirator tip of FIG. 1;

FIG. 3B is a cross-section view of a surgical aspirator tip end of the surgical aspirator tip of FIG. 2, taken across 3B-3B;

FIG. 3C is a side perspective view of the surgical aspirator tip end of FIG. 2;

FIG. 4A is a side perspective view of a surgical aspirator sleeve, where the sleeve is cut away to show the interior ribs and sleeve tip end projections;

FIG. 4B is a front view of a surgical aspirator sleeve;

FIG. 4C is an end view of the surgical aspirator sleeve;

FIG. 5A is a front view of a surgical aspirator tip joined with a surgical aspirator sleeve;

FIG. 5B is a cross-section view of the surgical aspirator tip end engaging the sleeve tip end projections, taken across 5B-5B;

FIG. 5C is a cross-section view of the surgical aspirator tip engaging the surgical aspirator sleeve, taken across 5C-5C;

FIG. 6 is a side perspective view of male coupling member joined to a female coupling member to form a coupled region of a surgical aspirator tip and sleeve combination;

FIG. 7A is a longitudinal cross-section view of an alternate embodiment of a surgical aspirator tip;

FIG. 7B is a cross-section view of a surgical aspirator tip end of the surgical aspirator tip of FIG. 7A, taken across 7B-7B;

FIG. 7C is a side perspective view of the surgical aspirator tip end of FIG. 7A;

FIG. 8 is a side perspective view of an alternate embodiment of a surgical aspirator sleeve, where the sleeve is cut away to show the interior pairs of converging longitudinal grooved ribs;

FIG. 9 is a cross-section view of the surgical aspirator tip end shown in FIG. 7A, wherein the tip end has engaged the converging grooved rib ends at the distal end of the aspirator sleeve;

FIG. 10 is a side perspective view of an alternate embodiment of the sleeve;

FIG. 11 is a side perspective view of an alternate embodiment of a male coupling member and grip member of a surgical aspirator tip;

FIG. 12 is a side elevational view of a surgical aspirator tip and a surgical aspirator sleeve; and

FIG. 13 is a side elevational view of a surgical aspirator tip joined with a surgical aspirator sleeve.

### DETAILED DESCRIPTION

Embodiments of a surgical aspirator tip and sleeve combination will now be described with reference to the drawings where like numerals correspond to like elements. Although embodiments of the present disclosure will be depicted generally as Yankauer or Andrews aspirator tips, one skilled in the relevant art will appreciate that the disclosed embodiments are illustrative in nature, and therefore, should not be construed as limited to application with either a Yankauer or Andrews tip. It should therefore be apparent that the embodiments of the present disclosure have wide application, and may be used on any similar aspirator tip and sleeve combination, such as a Frazier aspirator tip and sleeve combination. Accordingly, the following descriptions and illustrations herein should be considered illustrative in nature, and not limiting the scope of the present disclosure, as claimed.

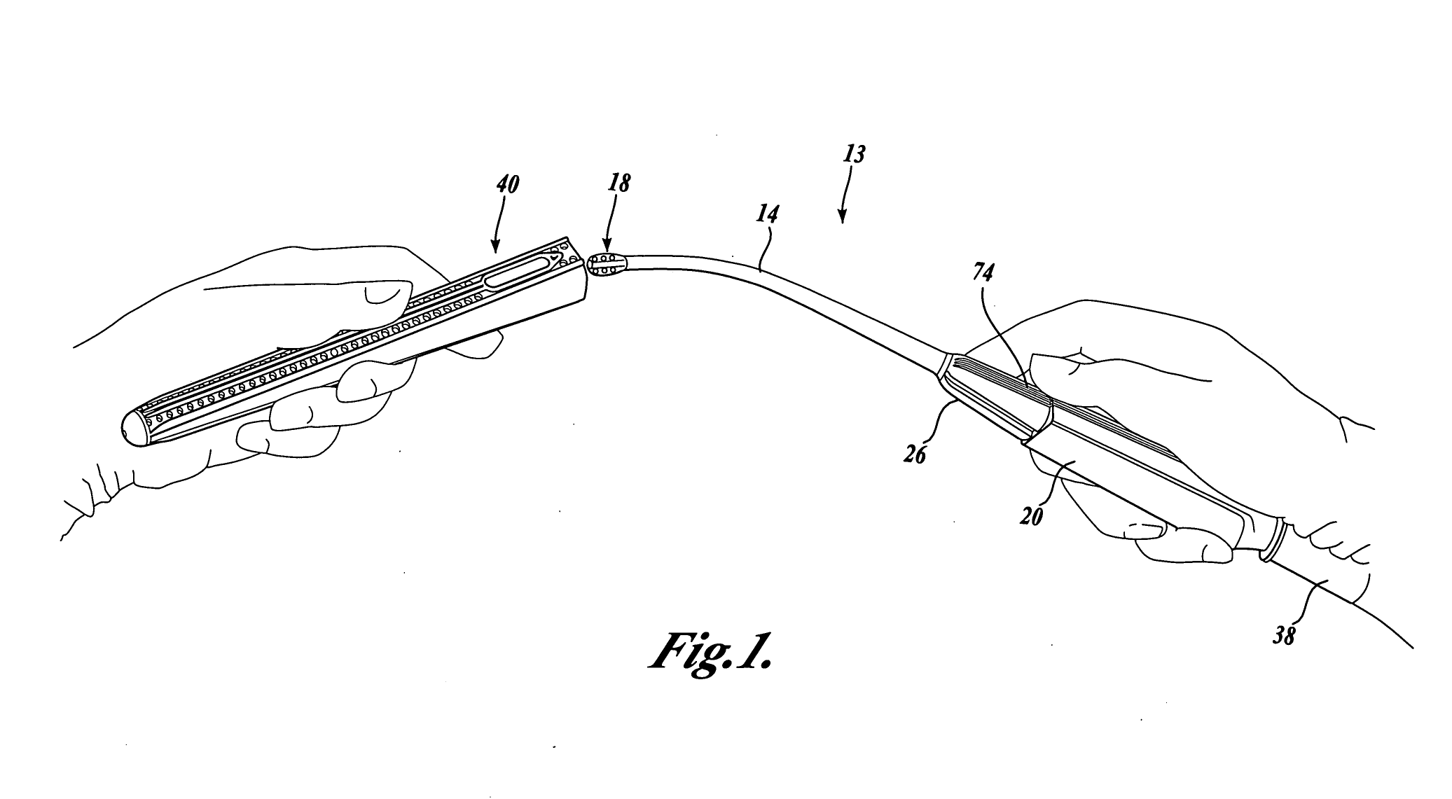

FIGS. 1-5 depict an embodiment of a surgical aspirator tip and sleeve combination. FIG. 1 shows a surgical aspirator tip 13, which may be received into a surgical aspirator sleeve 40 to form the surgical aspirator tip and sleeve combination.

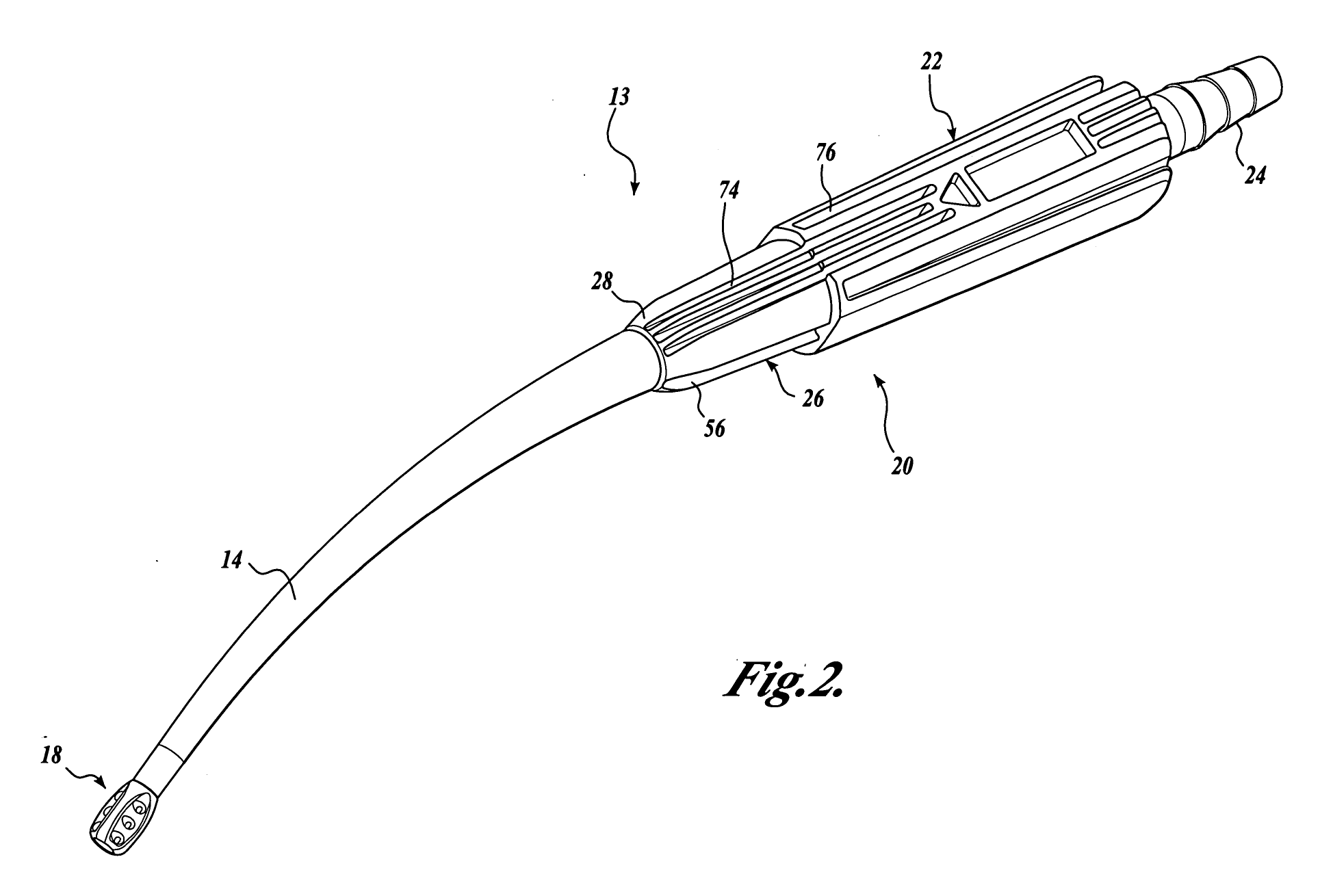

FIG. 2 depicts the surgical aspirator tip 13. The tip 13 generally includes a hollow tubular neck member 14 that is inserted into the wound, bodily orifice, or surgical site, and an enlarged medial section, or elongated handle member 20. The handle member 20 includes a grip section or member 22 for gripping the tip 13, a tube coupling member 24 that is used to attach the tip 13 to a tube 38 (depicted in FIG. 1) that in turn is connected to a source of suction (not shown), and a male coupling member 26 for attaching a sleeve 40 (see FIGS. 1) to the tip 13. The handle member 20 and tubular neck member 14 are constructed from a rigid or semi-rigid, resiliently deformable material that is adaptable for use in the medical arts. Preferably, polymeric or resinous plastic is used.

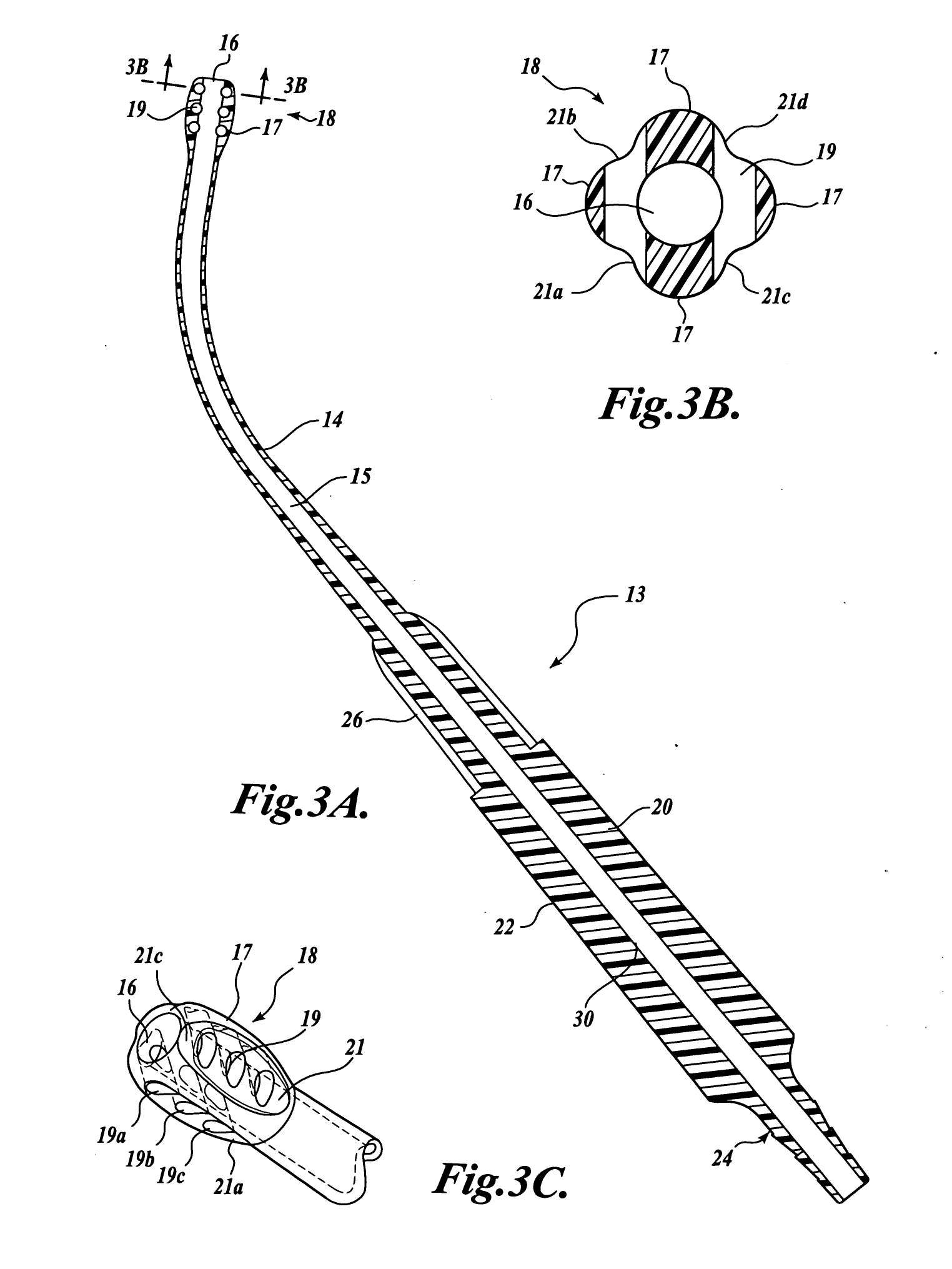

FIG. 3A depicts a cross-sectional view of the surgical aspirator tip 13. The handle member 20 defines a longitudinal internal channel 30. The proximal end of the tubular neck member 14 is attached to the distal end of the handle member 20 so that the interior 15 of the tubular neck member 14 is in communication with the internal channel 30 in the handle member 20.

As shown in FIGS. 3A-3C, an enlarged tip end portion 18, open at its distal end, is formed on the distal end of the tubular neck member 14. The tip end portion 18 defines a tip end opening or orifice 16 into which gases, fluids, and materials can flow. The tip end portion 18 is formed with tip end projections or ridges 17 that extend along the tip end portion 18. The tip end ridges 17 form tip end grooves 21 therebetween. The tip end portion 18 is preferably formed with four tip end ridges 17 that are generally the same size and shape and equidistant from one another, each ridge 17 being diametrically opposite another ridge 17. The tip end ridges 17 are used to abut the sleeve 40 to form a gap between the tip end portion 18 and the sleeve 40. However, if the tip 13 is used without the sleeve 40, the tip end ridges 17 are capable of bridging the adjacent soft tissue and maintaining the channels in the grooves 21 open for the flow of fluid, gas, and materials through the channels.

The tip end portion 18 may include additional tip end apertures or orifices 19. The tip end apertures 19 are formed in tip end grooves 21, and each tip end aperture 19 extends laterally through the tip end portion from a first tip end groove 21 to an adjacent tip end groove 21. FIGS. 3A-3C illustrate three rows of tip end apertures 19a-19c formed between adjacent tip end grooves 21a and 21b, and three rows of tip end apertures 19a-19c formed between adjacent tip end grooves 21c and 21d. Each row of tip end apertures 19a-19c is positioned substantially parallel to the other rows. The tip end apertures 19 intersect the tip end opening 16, such that the tip end apertures 19 are in communication with the tip end opening 16. In this manner, gases, fluids, and materials may flow within the grooves 21, through the tip end orifices 19, and into the opening 16 in the distal end of the neck portion 14. Although 3 rows of apertures are shown, it is to be understood that other numbers of rows of apertures 19, either fewer or greater in number, can be utilized. Also, the apertures are shown as round in cross-section, but the apertures can be of other cross-sectional shapes, such as oval, hexagonal, octagonal, etc.

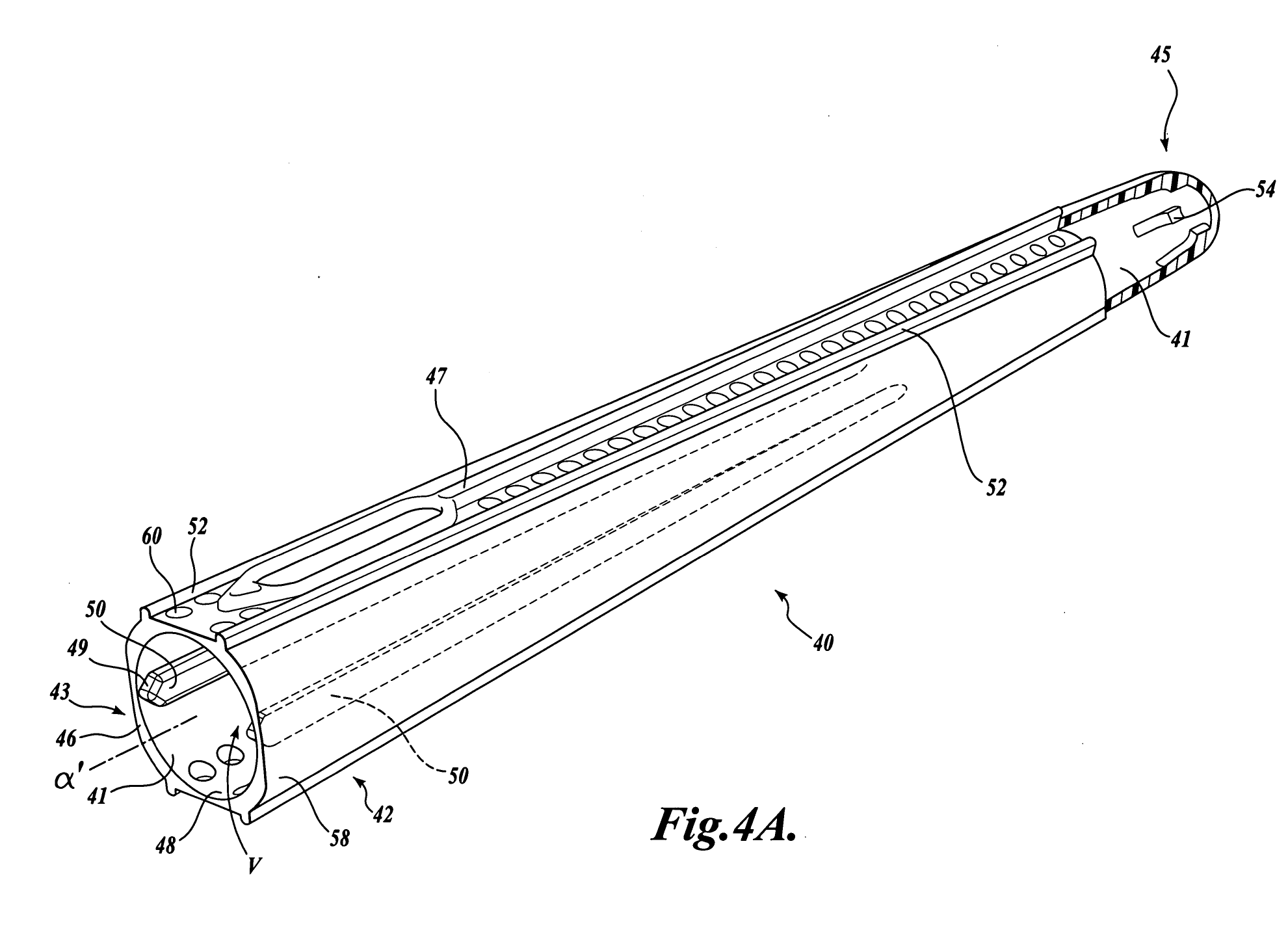

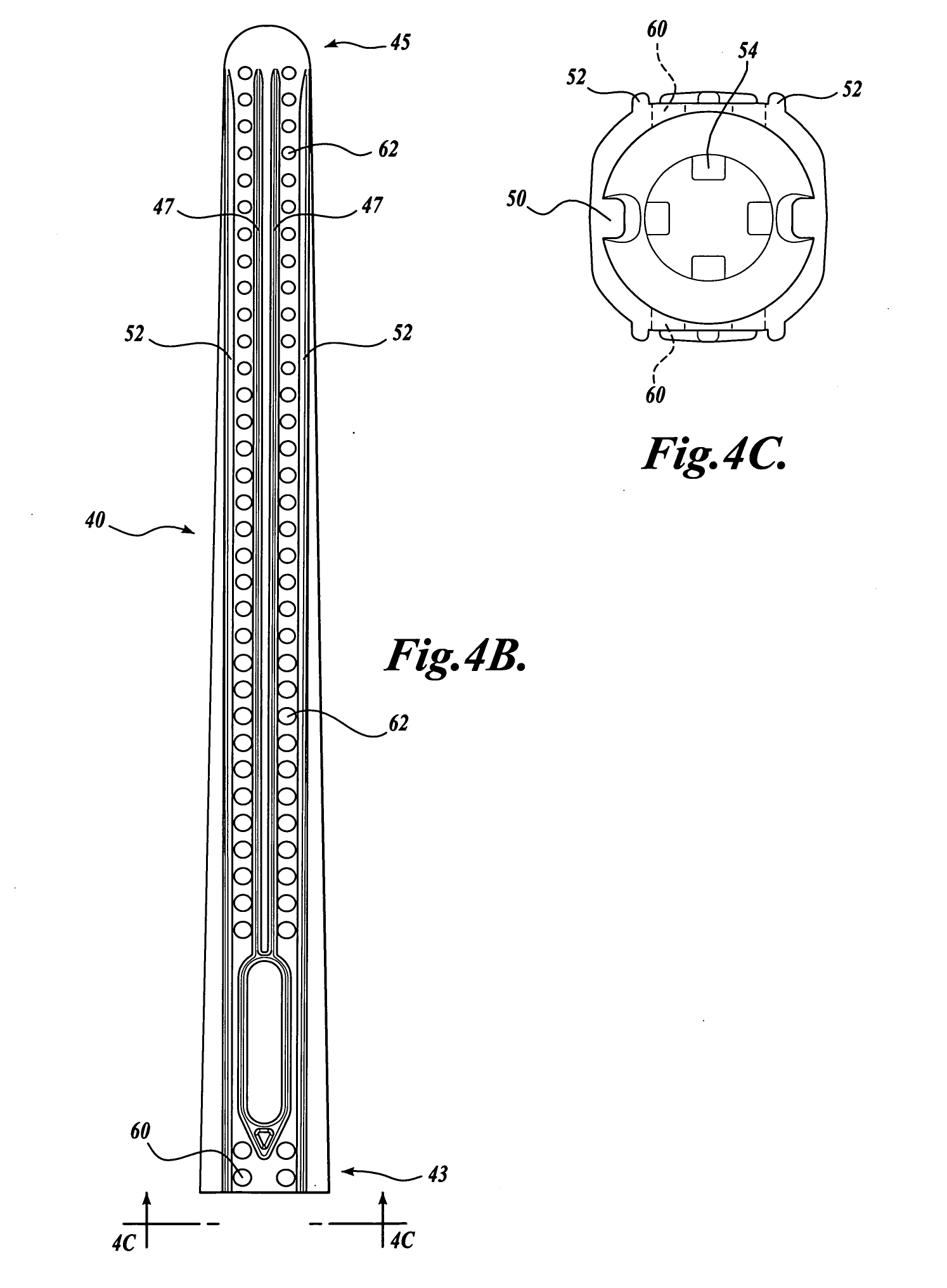

Now referring to FIGS. 4A, 4C, 5B, and sleeve 40 may include sleeve tip end projections 54 that protrude from the interior surface of the sleeve 41 at the distal tip sleeve end portion 45. Four sleeve tip projections 54 are shown as formed and configured to abut the four tip end ridges 17 when the tip 13 is received by the sleeve 40, as shown in FIG. 5B. When the tip end ridges 17 abut the sleeve tip end projections 54, a gap is formed between the sleeve interior surface 41 and the tip end portion 18. Thus, gas, fluid, and debris may freely flow into the sleeve 40, up towards the tip end portion 18, and into the tip end openings 16 and/or apertures 19.

Referring to FIG. 4A and 4B, the sleeve 40 may include grooves or ridges along its external surface as desired to aid in attaching or removing the sleeve 40. Preferably, sleeve exterior surface 58 includes ridges 47 and 52 that extend longitudinally along the length of the sleeve 40 on both the upper and lower surfaces of the sleeve 40. Optimally, two center ridges 47 are formed proximally to one another along the center of both the upper and lower surfaces of the sleeve 40, wherein such center ridges 47 are disposed between two lateral ridges 52. The sleeve 40 may include additional ribs, ridges, and other projections as well as grooves and depressions on the sleeve exterior surface 58 to lend structural support and aid in conducting gases, fluids, and materials into the interior of the sleeve 40.

The sleeve 40 includes an elongate, nominally straight sleeve tubular body that defines an internal channel having an open, proximal sleeve end portion 43 and an enclosed distal tip sleeve end portion 45. The sleeve 40 also contains a plurality of spaced orifices 62 that allow gases, fluids, and materials to flow into the interior of the sleeve 40. The orifices 62 are preferably round or ovoid but other shapes may be used. The orifices 62 are sized to permit the inflow of gases, fluids, and materials of a size that will not clog the opening 16 in the neck member 14 when the neck member is enclosed by the sleeve 40. Larger materials, on the other hand, such as body tissue, are unable to pass through the orifices 62 and may clog them. Thus, it is preferred, but not essential, that the orifices 62 are formed between the center ridges 47 and the lateral ridges 52 on each side of the sleeve 40 so that the ridges 47 and 52 may engage the tissue and form a gap between the tissue and the orifices 62, thereby preventing clogging. The orifices 62 on one side of sleeve 40 are in alignment with orifices 62 on the opposite side of the sleeve.

The sleeve 40 is preferably constructed from a material suitably flexible to conform to the shape of an aspirator neck 14 member inserted therein. Suitable materials to construct the tapered neck include rigid or semi-rigid, resiliently deformable materials adaptable for use in the medical arts such as polymeric or resinous plastic. The sleeve 40 may instead be contoured to match the contours present in the neck member 14.

Referring back to FIG. 2, the male coupling member 26 includes an outside surface 28. The male coupling member 26 may be formed in the distal portion of the handle member 20 or attached to the handle member 20 as a separate component. Alternatively, the male coupling member 26 may be attached to the neck member 14 and not attached to the handle member 20. The male coupling member 26 is between about [40 and 55 mm] long in the longitudinal α′ direction.

In one embodiment, the male coupling member 26 is generally tapered along its longitudinal axis α′ so that the cross-sectional area of the proximal end is greater than the cross sectional area of the distal end. In alternate embodiments, the cross-sectional areas of the proximal and distal ends may be approximately equal. Along its lateral axis, the proximal end of the male coupling member 26 is between about [4 and 20 mm] and the distal end is between about [4 and 20 mm]. In addition, the proximal cross-sectional area of the male coupling member 26 is less than the cross-sectional area of the distal end of the grip member 22.

The cross-sectional shape of the male coupling member 26 may remain constant or vary (as depicted in FIG. 2) along the longitudinal axis α. The male coupling member 26, excluding longitudinal exterior grooves 74 (described below), may have any cross-sectional shape, but preferably has a cross-sectional shape that is generally round, ovoid, square, rectangular, triangular, hexagonal, or other closed shape. To aid in attaching or removing the sleeve 40, the male coupling member 26 may also include ridges or grooves along the length of male coupling member 26.

Handle member 20 includes at least one longitudinal exterior groove 74 extending longitudinally along the outside surface of handle member 20. In one embodiment, longitudinal exterior grooves 74 extend from the distal to the proximal end of male coupling member 26, but it is appreciated that the grooves 74 may extend from the proximal end of the male coupling member 26 and along only a portion of the male coupling member 26. In the alternative, longitudinal exterior grooves 74 may extend onto a section of the grip member 22 from its distal end. Alternatively, separate grooves may be included in the grip member 22 that are in communication or intersect with longitudinal exterior grooves 74 on the male coupling member 26. Longitudinal exterior grooves 74 are between 1 and 7 mm deep and 1 and 10 mm wide, and have any cross-sectional shape such as U-shaped, V-shaped or other suitable groove shape. Three longitudinal exterior grooves 74 are shown as formed on the male coupling member 26 that extend longitudinally along the entire length of the handle member 20, at the upper and lower sides of the handle members. In addition, a lateral groove 76 may be formed on each lateral side of longitudinal exterior grooves 74 to further enhance the grip and aid in attaching or removing the sleeve 40.

Still referring to FIG. 2, the grip member 22 is suitably sized to be received into an average sized hand but larger or smaller grip sections may be constructed for larger or smaller hands respectively. Generally, the grip member 22 may be between about [35 and 80 mm] long and have a cross-sectional width between about [12 and 30 mm] and a cross-sectional height between about [12 and 30 mm]. The grip member 22 may also be tapered or include contours along its longitudinal axis for a more comfortable grip.

Referring back to FIG. 4A, the sleeve 40 includes an open, proximal aspirator sleeve end, or female coupling portion 42, and an enclosed, distal tip sleeve end portion 45. The female coupling member 42 includes a wall 46 with an internal surface 48 that defines an internal receiving volume V. The female coupling member 42 is generally tapered along its longitudinal axis α′ so that the cross-sectional area of the proximal end is greater than the cross-sectional area of the distal end. In alternate embodiments, other profiles may be used such that the cross-sectional areas of the female coupling portion 42 and the distal section 45 of the sleeve 40 are approximately equal. In another embodiment, the female coupling portion 42 is tapered or contoured to approximate the taper or contour of the male coupling member 26. Along its lateral axis, the proximal end of the female coupling member 42 is between about [8 and 24 mm] and the distal end is between about [8 and 24 mm].

The cross-sectional shape of the female coupling member 42 of the sleeve 40 may remain constant or vary along the longitudinal axis α′. The female coupling member 42 of the sleeve 40 may have any cross-sectional shape but is preferably generally round, ovoid, square, rectangular, triangular, hexagonal, or other closed shape. In an alternate embodiment, the cross-sectional shape of the female coupling portion 42 approximates the cross-sectional shape of the male coupling member 26.

Referring now to FIGS. 5A-5C, the sleeve 40 slides over the neck 14 of the tip 13 so that the neck 14 is completely encased by the sleeve 40. Generally, the sleeve 40 is attached to the tip 13 at the handle member 20 by a coupling device. The coupling device includes a tip coupling member such as the male coupling member 26, shown in FIG. 2, and a sleeve coupling member such as the female coupling member 42, shown in FIG. 4A. The male coupling member is received into the receiving volume V (see FIG. 4A) of the female coupling member 42. A coupled region 70 is formed where the male coupling member 26 is inserted into the female coupling member 42.

Referring again to FIG. 2, sleeve alignment grooves 56 may be formed on the male coupling member 26. The sleeve alignment grooves 56 are formed in the proximal end of the male coupling member 26, and extend a predetermined distance towards the distal end of the male coupling member 26. The sleeve alignment grooves 56 are formed on opposite sides of the male coupling member 26 on portions on the male coupling member outside surface 28 not covered by longitudinal exterior grooves 74. The sleeve alignment grooves 56 may have any cross-sectional shape, but preferably have a cross-sectional shape that is generally U-shaped, V-shaped, or other suitable groove shape.

Referring back to FIG. 4A, sleeve 40 may include sleeve alignment ribs 50 formed along a portion of the interior surface of the sleeve 41 in the space between the orifices 62. The sleeve alignment ribs 50 extend from the proximal end of the sleeve 43 towards the distal tip sleeve end portion 45. Preferably, two sleeve alignment ribs 50 are formed on the interior surface of the sleeve 41 on opposite sides of the sleeve 40. The sleeve alignment ribs 50 taper in height as the ribs 50 extend toward the distal tip sleeve end portion 45. The sleeve alignment ribs 50 substantially conform to the shape of the sleeve alignment grooves 56, such that the sleeve alignment grooves 56 may closely, slidably receive the sleeve alignment ribs 50 when the sleeve 40 receives the tip 13, as shown in FIG. 5C. The sleeve alignment ribs 50 are tapered at the proximal end of the sleeve to form lead-in portions 49. The lead-in portions 49 aid in securing the sleeve 40 to the tip 13 by guiding the sleeve alignment ribs 50 into the sleeve alignment grooves 56.

The sleeve alignment ribs 50 are slidably received by the sleeve alignment grooves 56 so that the sleeve 40 is properly aligned and coupled to the tip 13. When properly mated, the tip end projections 54 abut the four tip end ridges 17 to form a gap between the tip end portion 18 and the sleeve 40, as shown in FIG. 5B. Additionally, cross-holes 60 remain properly aligned with longitudinal exterior grooves 74 (as described) to ensure proper venting and air flow into the sleeve 40. Moreover, when the ribs 50 are slidably received by the grooves 56, the sleeve 40 is locked into place and will not rotate about tip 13. Thus, while the tip 13 is being used, the tip end projections 54 will remain abutted to the four tip end ridges 17, and the cross-holes 60 will remain properly aligned with longitudinal exterior grooves 74.

To further aid in proper alignment, an indicator design or indicia 59 may be formed on the sleeve 40 and handle member 20. Preferably, the indicator design or indicia 59 comprises an arrow or other suitable design or indicia. The indicator design 59 is formed on the center ridges 47 of the sleeve 40 in the form of an arrow, with the arrow pointing towards the proximal end of the sleeve 40. The indicator design 59 is formed on both sides of the sleeve 40. A similar design is formed on the on the longitudinal exterior grooves 74 of the grip section 22, with the arrow pointing towards the male coupling member 26. Either arrow on the sleeve 40 may be aligned with the arrow on the grip member 22 when inserting the tip 13 into the sleeve 40, such that the sleeve may be rotated 180° and still properly mate with the tip. The indicator designs 59 will facilitate proper alignment of the sleeve alignment ribs 50 with the sleeve alignment grooves 56, thereby ensuring that the tip end projections 54 abut the four tip end ridges 17. It should be appreciated that any suitable design or indicia may be used to guide the insertion of the tip 13 into the sleeve 40.

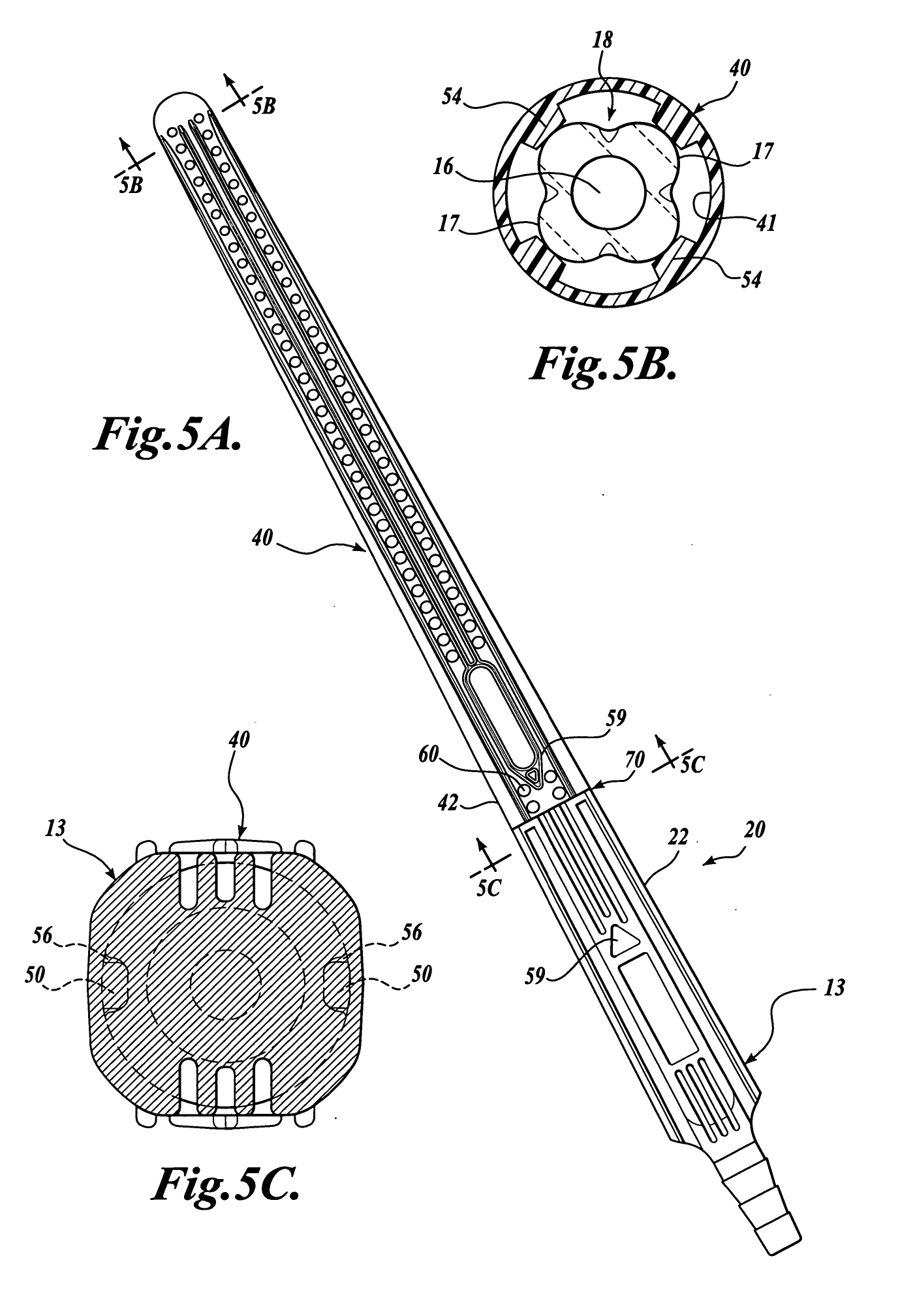

Now referring to FIG. 6, the internal surface 48 of the wall 46 of the female coupling member 42 contacts the outside surface 28 of the male coupling member 26 along the coupled region 70. Portions of the internal surface 48 of the female coupling member 42 do not contact the outside surface 28 of the male coupling member 26. Particularly, the sections of the internal surface 48 of the female coupling member 42 adjacent to longitudinal exterior grooves 74 do not contact the outside surface 28 of male coupling member 26. Consequently, venting channels 80 are formed between the internal surface 48 of the wall 46 and the outside surface 28 of the male coupling member 26 as depicted in FIG. 6. These venting channels 80 allow air to flow between the external environment into the interior of the sleeve 40. Each individual venting channel is in communication with other venting channels, the external environment, and/or the interior of the sleeve 40 as required to provide communication between the external environment and the interior of the sleeve 40.

With the sleeve 40 in place, the distal end of the tip 13 and sleeve 40 combination may be inserted into the wound, surgical site, or bodily orifice to remove fluids therein. Suction flows from the suction source, such as a suction pump, through the tube 38 and into the handle member 20 (as shown in FIG. 1). As shown in FIG. 2, the tube coupling member 24 may include a tiered section that is coupled to the tube 38 (see FIG. 1) by inserting one or more of the tiers having a smaller cross-sectional area into the tube 38, however, any tube coupling mechanism may be used. The tube 38 may be constructed from any tubular material suitable for transmitting suction forces to a surgical aspirator and gases, fluids and materials from a surgical site known in the medical arts.

Suction traverses the handle member 20 and into the neck member 14. Suction travels up the neck and pulls gases, fluids, and small materials into the opening 16. The gases, fluids, and materials inside the sleeve 40 flow from the wound, surgical site, or bodily orifice into the sleeve 40 through the plurality of orifices 62 and opening 16. If the orifices 62 become clogged such that the flow of gases, fluids, and materials into the interior of the sleeve 40 is restricted, air flow is available to the sleeve through the venting channels 80. Air provided by the venting channels may prevent uneven distribution of suction forces over any unclogged orifices 62. Otherwise, the suction force is concentrated over too few orifices 62, the tissue surrounding the wound, surgical site, or orifice could be pulled into the orifices 62 in the sleeve 40 possibly causing discomfort, pain, and injury to the patient.

The tip 13 may be used without the sleeve 40 to accurately and efficiently drain fluids from a specific area, such a surgical site. Accurate and effective draining is necessary because even a small amount of fluid or film can obstruct a medical operator's view. When placing the tip end portion 18 within a body cavity, the tip end ridges 17 bridge the adjacent soft tissue and maintain the channels open in the grooves 21. Thus, if the tip end opening 16 is clogged, fluid, gas, and materials may flow into the channels defined by grooves 21 and into the openings 19. If the tip 13 is placed within a cavity so that is oriented substantially orthogonally to a tissue wall, the tip end opening 16, as well as the openings 19 adjacent the end opening, may be clogged with tissue. In this case, the fluid, gas, and materials may flow into the channels defined by grooves 21 and into the uncovered openings 19 located father away form the opening 16.

In one embodiment depicted in FIGS. 5A and 6, the distal end of the grip member 22 abuts the proximal end of the female coupling member 42. As mentioned above, longitudinal exterior grooves 74 extend onto grip member 22 from the distal end. Air flows through the portion of longitudinal exterior grooves 74 located in handle member 22 into the venting channels 80. This configuration may prevent both the hands of the user and the distal end of the grip member 22 from interfering with the air flow through the venting channels 80.

Referring to FIG. 6, cross-holes 60 may be formed in the portion of the wall 46 between the venting channels 80 and the external environment to provide another means by which air may enter the venting channels 80. Because the handle 20 is not generally in contact with the tissue or fluids at the surgical site, the portion of longitudinal exterior grooves 74 located in the handle member 22 and cross-holes 60 in the sleeve 40 are unlikely to become clogged with tissue. Therefore, a constant airflow is available inside the sleeve 40 and particularly in the area surrounding opening 16 in the tip 13. This airflow prevents uneven distribution of suction to the holes of the sleeve 40.

As a non-limiting example, the tip 13 of the present invention may be formed by injection molding. For illustrative purposes, one non-limiting example of a method by which the tip 13 may be constructed will be provided herein.

A tip mold is first formed to produce a complete tip 13 during the molding process. The tip guard mold includes an upper and lower portion, and each portion of the tip guard mold contains a portion of a mold cavity. The upper and lower portions contain a mold for the upper half of the tip 13 and the lower half of the tip 13. Further, the mold includes inwardly extending projections that extend into the mold cavity to form additional openings and grooves in the tip 13.

Both portions of the tip guard mold are coupled together to define the mold cavity therebetween. At least one inlet channel is included in the mold to allow the inflow of material into the mold cavity. An injection nozzle injects material through the inlet channel and into the mold cavity. The injected material fills the mold cavity and surrounds a section of the tip guard core.

While one method of forming the tip is depicted in this application, it is apparent to one of ordinary skill in the art that alternate equivalent methods are available. For example, both the handle member 20 and the tubular neck member 14 could be molded separately and then secured together with a fluid tight seal.

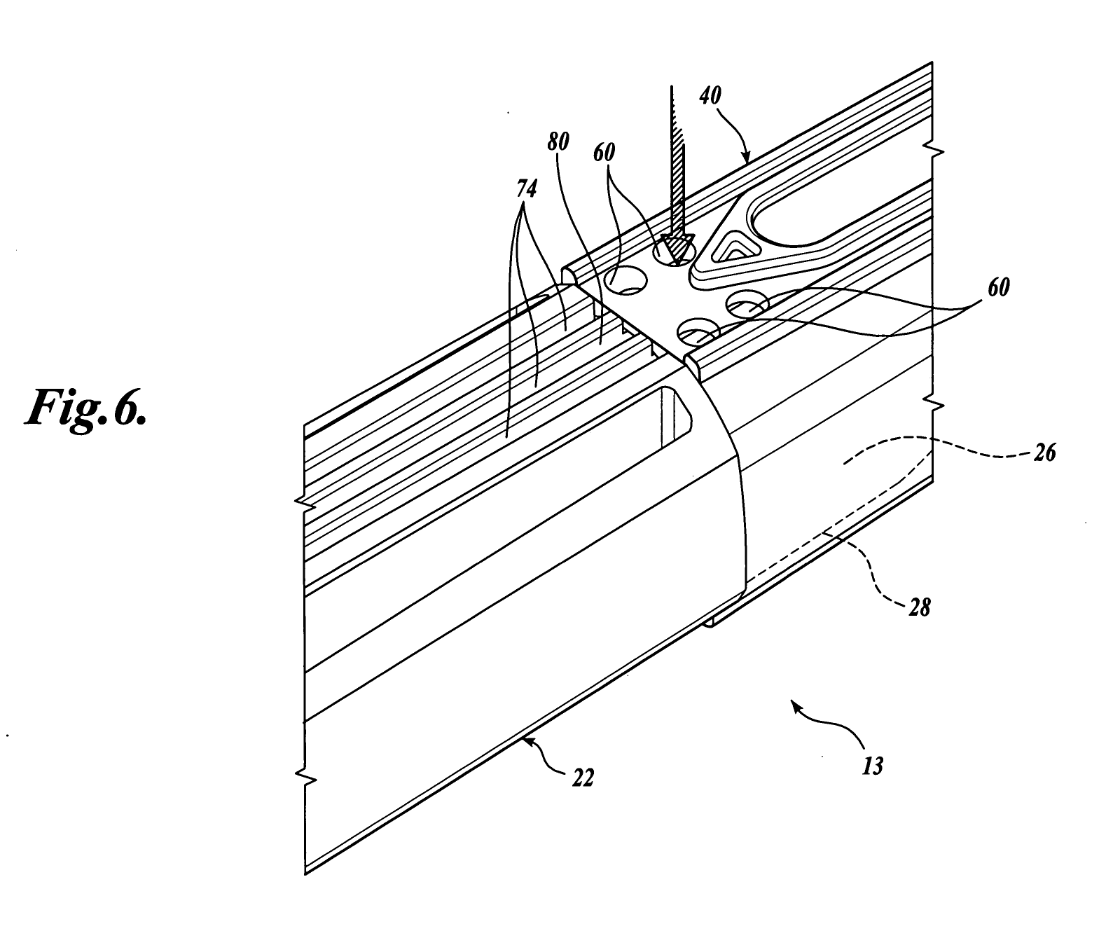

Referring now to FIGS. 7-12, an alternate embodiment of the present invention is depicted, wherein like numerals are used for like parts relative to FIGS. 1-6. Referring first to FIG. 7A-7C, the tip end portion 118 contains tip end projections 117 that extend transversely from the tip end portion 118, as shown in FIG. 7C. The tip end projections 117 are used to secure the tip end portion 118 of the tip 110 within the distal end of the sleeve 140. Preferably, the tip end portion 118 contains four tip end projections 117 that are generally the same size and shape and equidistant from one another. Alternatively, two symmetrical tip end projections 117 with two tip end grooves 121 may be used. The tip end portion 118 includes additional tip end orifices 119 located circumferentially about tip end portion 118, which extend from the exterior surface of the tip end portion 118 radially through the tip end portion 118 intersecting the tip end opening 116, as shown in FIG. 7B. The tip end orifices 119 are preferably disposed between the tip end projections 117. Therefore, four tip end orifices 119 may be formed, but it may be appreciated that greater or less than four tip end orifices 119 may be included.

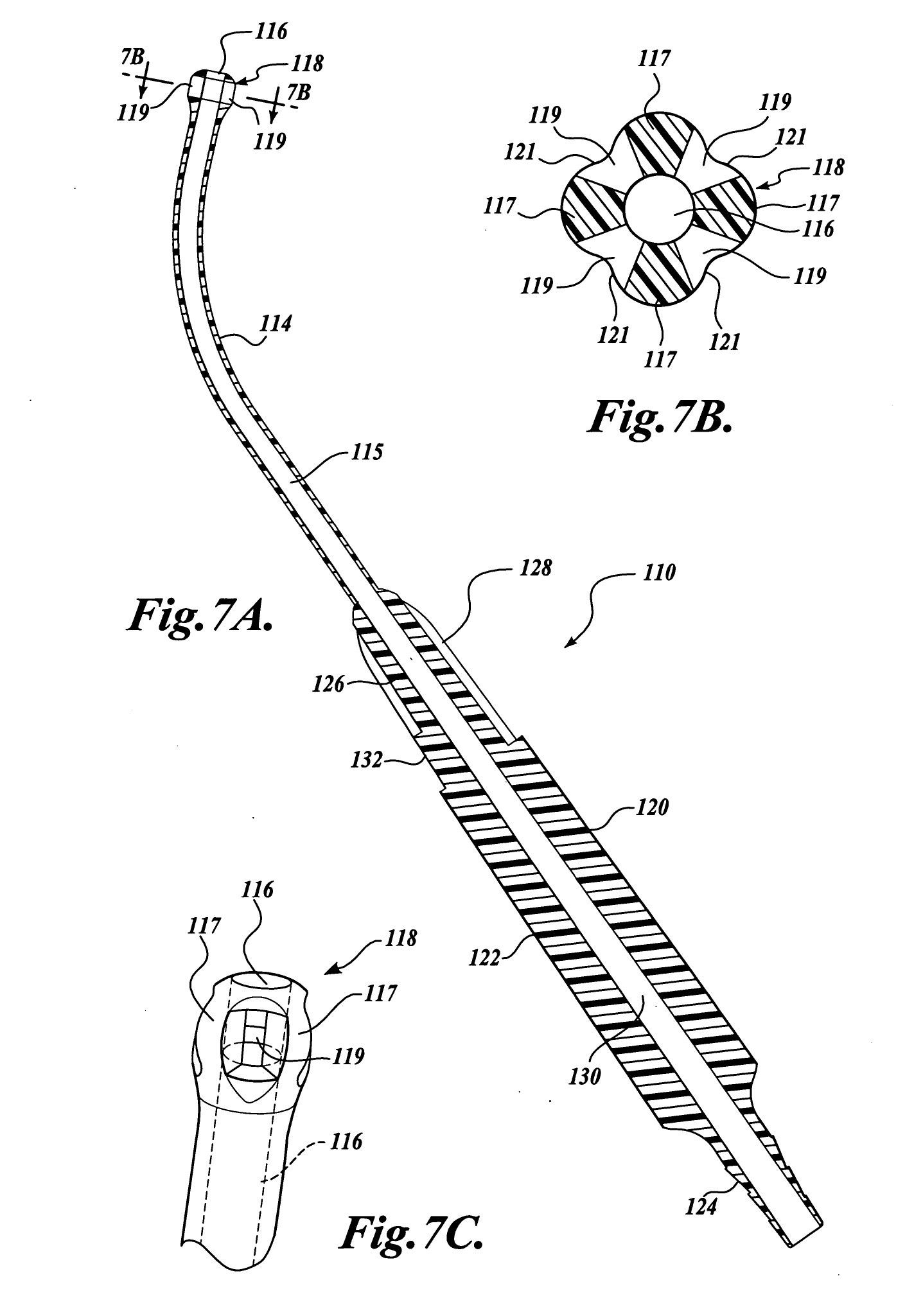

Referring now to FIG. 8, a sleeve 140 includes at least two pairs of converging longitudinal grooved ribs 150 on the interior surface 141 of the sleeve 140. The pairs of grooved ribs 150 originate at the proximal end of the sleeve 140 and converge near the distal tip sleeve end portion 145 of the sleeve 140 to form a converged rib end 151. In another embodiment, the grooved ribs 150 may originate at the distal end of the female coupling member 142 and converge near the distal tip sleeve end portion 145. The sleeve 140 is shown as having four pairs of converging longitudinal grooved ribs 150 on the interior surface 141 of the sleeve 140. Each pair of converging longitudinal grooved ribs 150 are spaced generally equidistant from the other grooved ribs 150.

The converging longitudinal grooved ribs 150 protrude from the interior surface 141 of the sleeve 140 such that the ribs 150 may contact the tubular neck member 114 of the tip 110 where the sleeve 40 has flexibly conformed to the shape of the neck 114. Thus, where the neck member 114 is bent, the sleeve 140 engages the neck 114 when the sleeve 140 bends to generally conform to the shape of the neck 114. In those areas, the ribs 150 may engage the neck 114 to maintain a gap between the neck 114 and the sleeve 140 and to allow the passage of fluids and other debris.

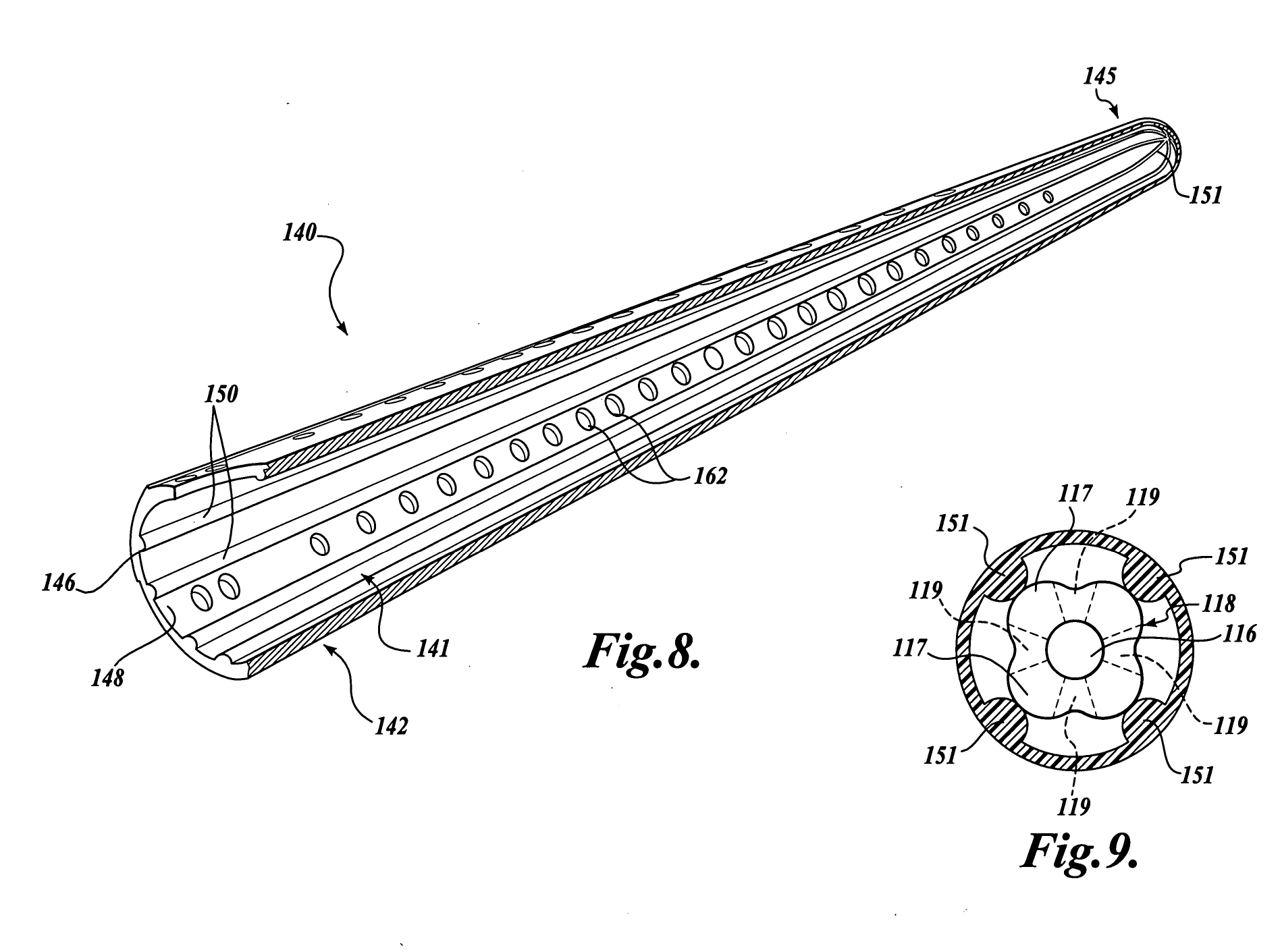

Referring to FIG. 9, the tip end projections 117 on the tip end portion 118 engage the converged rib ends 151 at the distal tip sleeve end portion 145 when the neck member 114 is inserted into the sleeve 140. The converged rib ends 151 abut the tip end projections 117, forming a predetermined gap between the interior of the distal tip sleeve end portion 145 and the tip end portion 118. This gap may enable gas, fluids, and other debris to flow more freely into the tip end orifices 119 and the tip end opening 116 after entering the sleeve 140.

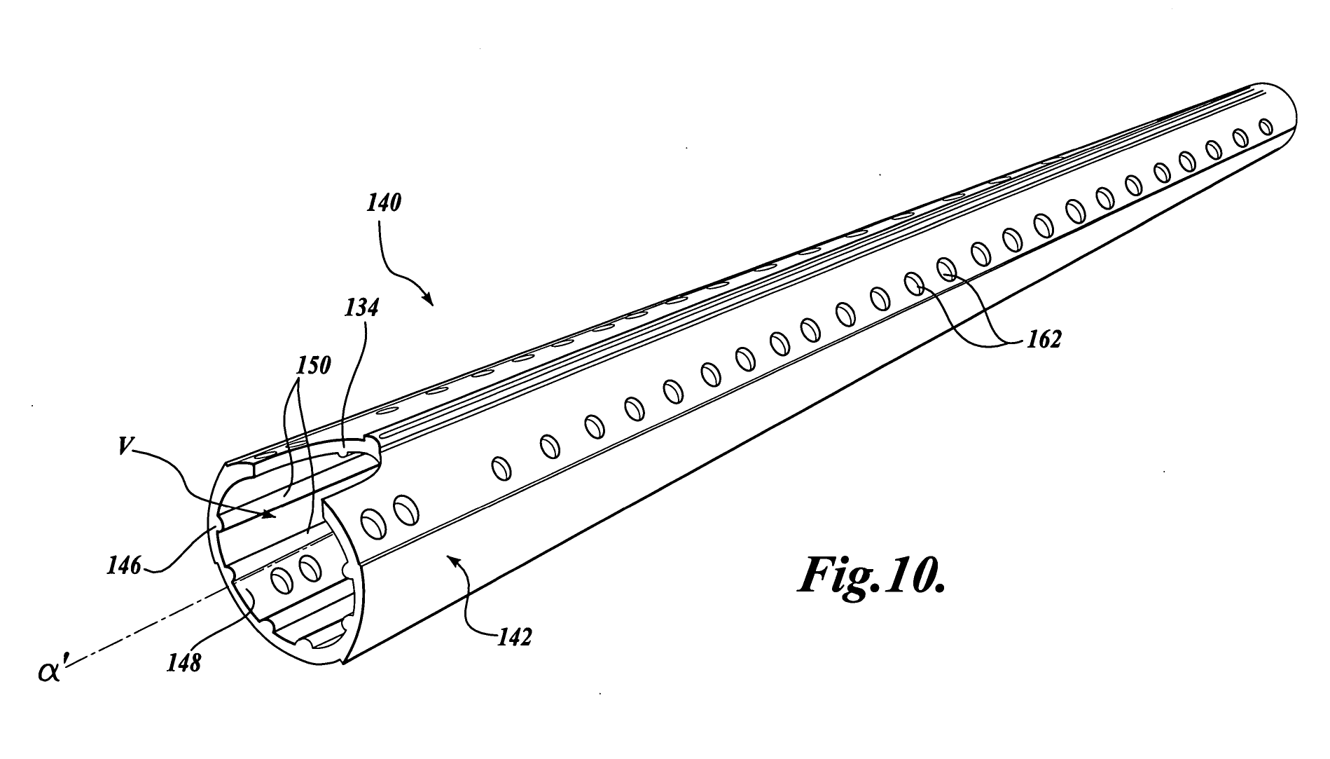

Referring to FIGS. 10-13, a key and slot joint 131 is used to secure the coupled region 170. A slot 134 is formed in the proximal portion of the female coupling member 142, as shown in FIG. 10. The slot 134 is preferably U-shaped; however, other shapes may also be used. Moreover, the slot 134 is preferably formed in only a portion of the female coupling member 142. In other words, the slot 134 preferably does not extend from the proximal portion of the female coupling member 142 to the distal portion of the female coupling member 142. However, in alternate embodiments, the slot 134 may extend along the entire length of the female coupling member 142.

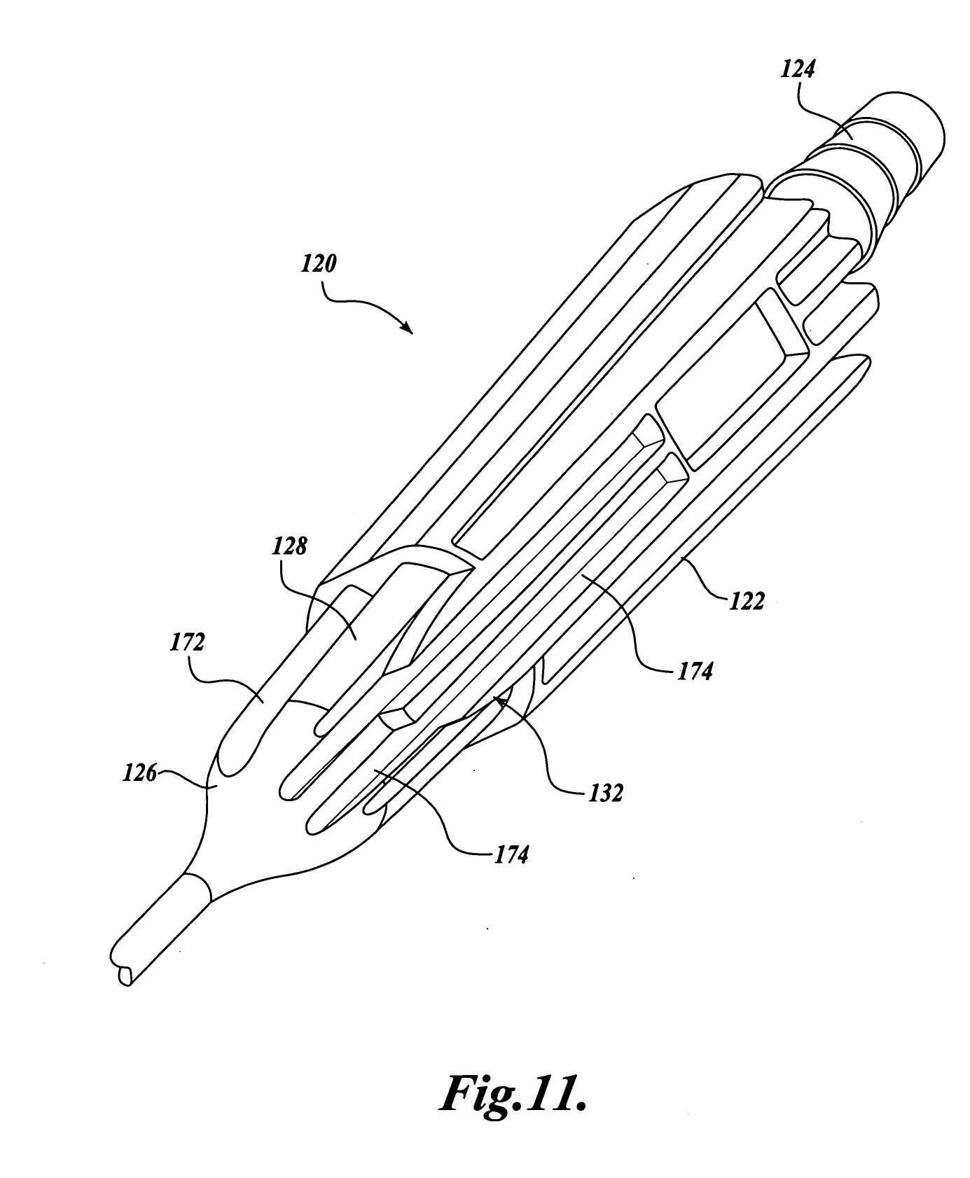

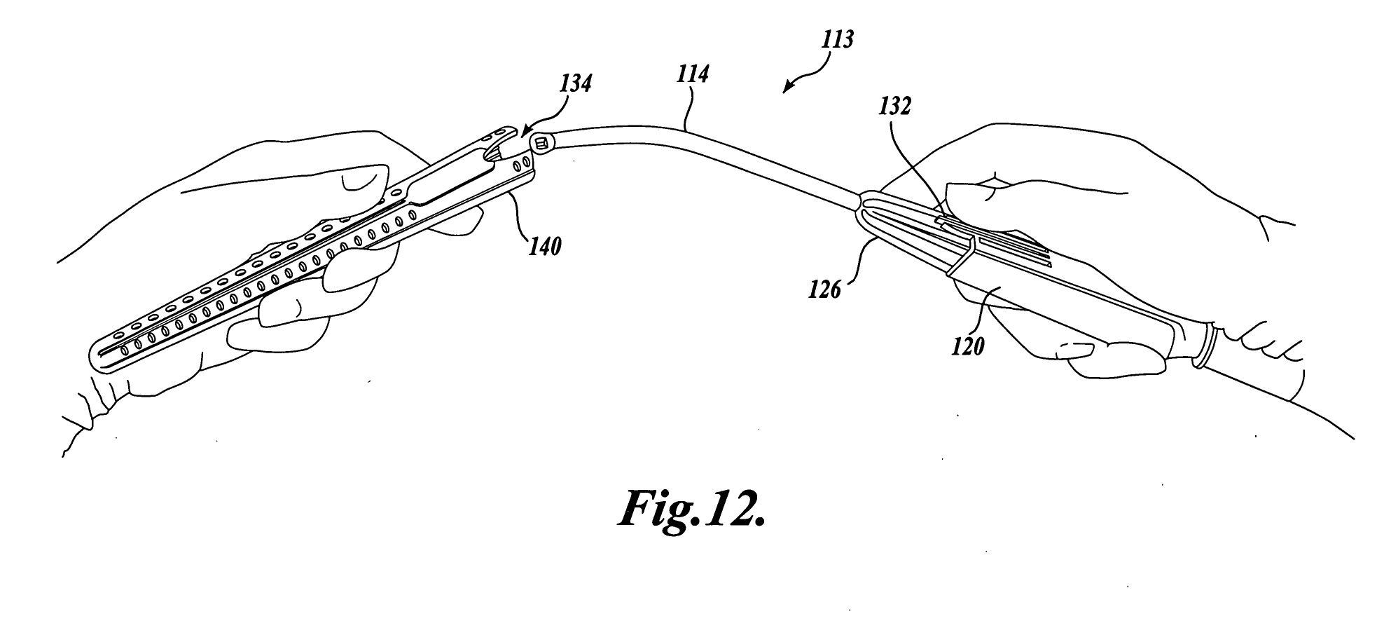

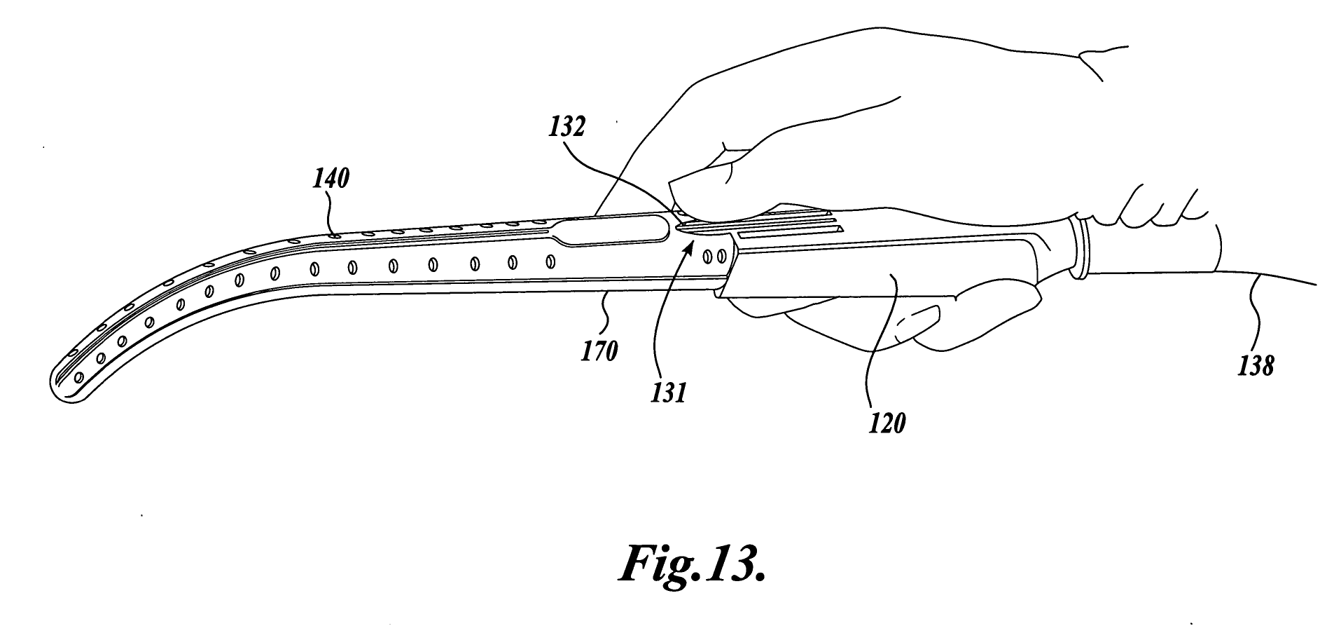

A key 132 is formed near the proximal portion of the male coupling member 126 on the male coupling member outside surface 128, as shown in FIG. 11. The longitudinal exterior grooves 174 may still be formed on the section of the male coupling member 126 where the key 132 is formed. In the alternative, the longitudinal exterior grooves 174 may not be formed on the key 132, such that the key 132 is solid. The key 132 is sized and shaped to generally conform to the size and shape of the slot 134. The key 132 protrudes from the male coupling member outside surface 128, such that the thickness of the key 132 is equal to or slightly greater than the thickness of the sleeve 140. Thus, when the male coupling member 126 is received into the female coupling member 142, as depicted in FIGS. 12 and 13, the slot 134 receives the key 132 to form the key and slot joint 131. The key and slot joint 31 ensures a proper fit between the male coupling member 126 and the female coupling member 142, such that the tip end projections 117 on the tip end portion 118 engage the converged rib ends 151 at the distal tip sleeve end portion 145 when the neck member 114 is inserted into the sleeve 140. In addition, the key and slot joint properly aligns the sleeve 140 with the tip 113 so that sleeve orifices 162 remain adjacent the longitudinal grooves 174 to ensure proper venting and air flow into the sleeve 140. The key and slot joint 131 may also lock the sleeve 140 into its position on the tip 113 and prevent the rotational movement of the female coupling member 142 relative to the male coupling member 126.

While the preferred embodiment of the invention has been illustrated and described, it will be appreciated that various changes can be made therein without departing from the spirit and scope of the invention.

1. A sleeve and aspirator tip combination comprising:
(a) a surgical aspirator tip comprising:
(i) an enlarged medial portion having an internal channel and an external medial portion surface, said medial portion including at least one longitudinal exterior groove formed on the external medial portion surface;
(ii) a hollow tubular neck member having an exterior surface, said hollow tubular neck member extending distally from the medial portion and with the hollow tubular neck member being diametrically no larger than the enlarged medial portion;
(iii) a tip end portion at the distal end of the hollow tubular neck member opposite the medial portion, said tip end portion defining at least one tip end opening that is in communication with the hollow tubular neck member;
(b) a surgical aspirator sleeve comprising:
(i) an elongate, tubular body composed of resilient deformable material, the body having an internal sleeve surface and an external sleeve surface;
(ii) said tubular body defining an internal channel having an open, proximal aspirator sleeve end portion and an enclosed, distal tip sleeve end portion, said tubular body defining plural, spaced orifices at a spaced distance from the distal tip sleeve end portion, said orifices providing communication between the external environment and internal channel of the tubular body to allow passage of gases, fluid, or materials to be aspirated into the internal channel defined by the tubular body;
(iii) said tubular body slidably receivable over the hollow tubular neck member of the surgical aspirator tip to assume the profile of the hollow tubular neck member of the aspirator tip;
(iv) wherein the size of the internal channel of said tubular body is sufficiently larger than the exterior of the hollow tubular neck member to enable the gases, fluid, or materials passing into the channel through the orifices to flow through the internal channel between the interior of the tubular body and the exterior of the hollow tubular neck member toward the distal tip end of the hollow tubular neck member; and
(c) at least one venting channel in communication with the interior of the sleeve and the external environment formed between the at least one longitudinal exterior groove of the enlarged medial portion and the internal surface of the proximal aspirator sleeve end, allowing airflow between the external environment and the interior of the sleeve;
(d) locking means to secure said tubular body to an aspirator tip and to resilient relative longitudinal and rotational movement of said tubular body with respect to the aspirator tip; and
(e) alignment means for guiding the sleeve onto the tip to properly mate the tubular body and aspirator tip, thereby ensuring the formation of at least one venting channel therebetween.

2. The sleeve and aspirator tip combination of claim 1, further comprising:
(a) at least two tip end projections formed on the tip end portion and extending generally laterally to the hollow tubular neck member, wherein the tip end projections form at least one tip end groove therebetween; and
(b) at least two tip end sleeve projections formed on the interior surface of the sleeve at the sleeve distal tip end portion, said projections being configured to selectively engage the tip end projections such that a gap is formed between the sleeve and the tip when the aspirator tip is received within the aspirator sleeve.

3. The sleeve and aspirator tip combination of claim 2, wherein the sleeve tip end projections formed on the interior surface of the sleeve at the sleeve distal tip end portion are defined by at least two pairs of converging longitudinal grooved ribs extending along at least a portion of the internal sleeve surface of the sleeve tubular body and converging near the distal tip end of the aspirator sleeve, wherein the tip end projections may selectively engage the tip end projections.

4. The sleeve and aspirator tip combination of claim 2, the tip end portion further comprising at least one tip end aperture formed in the tip end grooves, said at least one tip end aperture in communication with the tip end opening.

5. The sleeve and aspirator tip combination of claim 4, wherein the tip end projections are sized and located to bridge adjacent tissue when the tip is used without the sleeve so that fluids, gases and materials are capable of flowing within the tip end grooves and into the tip end apertures.

6. The sleeve and aspirator tip combination of claim 4, wherein the tip end apertures extend laterally to the tip end portion, the tip end apertures extending between adjacent tip end grooves and intersecting the tip end opening.

7. The sleeve and aspirator tip combination of claim 4, wherein the tip end apertures extend radially outwardly from the tip end opening through the tip end portion to the tip end grooves.

8. The sleeve and aspirator tip combination of claim 4, wherein the tip end apertures are located at different distances from the tip end opening.

9. The sleeve and aspirator tip combination of claim 1, wherein the open, proximal end portion of the tubular body of the aspirator sleeve is resiliently expandable.

10. The sleeve and aspirator tip combination of claim 1, wherein the internal channel of the enlarged medial portion is in communication with the interior of the hollow tubular neck member allowing gases, fluid, or materials to flow from the interior of the hollow neck member through the internal channel of the enlarged medial portion.

11. The sleeve and aspirator tip combination of claim 1, wherein the external sleeve surface has a plurality of outwardly projecting ribs.

12. The sleeve and aspirator tip combination of claim 1, wherein said locking comprises at least one groove formed on the exterior surface of the enlarged medial portion that slidably engages at least one rib formed on the interior surface of the sleeve tubular body.

13. The sleeve and aspirator tip combination of claim 1, wherein said locking means comprises a key and slot joint.

14. The sleeve and aspirator tip combination of claim 13, wherein the key and slot joint further comprises:
(a) a key protruding from at least a portion of the external medial portion surface;
(b) a slot formed in the proximal aspirator sleeve end portion, the slot generally conforming to the shape of the key; and
(c) wherein the key is engageable with the slot to restrain rotational movement of said sleeve tubular body relative to the aspirator tip.

15. The sleeve and aspirator tip combination of claim 1, wherein said alignment means for mating the tubular body to the aspirator tip comprises a key and slot joint.

16. The sleeve and aspirator tip combination of claim 15, wherein the key and slot joint further comprises:
(a) a key protruding from at least a portion of the external medial portion surface;
(b) a slot formed in the proximal aspirator sleeve end portion, the slot generally conforming to the shape of the key; and
(c) wherein the key is engageable with the slot to restrain rotational movement of said sleeve tubular body relative to the aspirator tip.

17. The sleeve and aspirator tip combination of claim 1, wherein said alignment means for mating the tubular body to the aspirator tip comprises at least one groove formed on the exterior surface of the enlarged medial portion that slidably engages at least one rib formed on the interior surface of the sleeve tubular body.

18. The sleeve and aspirator tip combination of claim 1, wherein said alignment means for mating the tubular body to the aspirator tip comprises at least a first indicia formed on a portion of the sleeve external surface and at least a second indicia formed on a portion of the enlarged medial portion, wherein the first indicia may be visually aligned with the second indicator when the tip is being slidably received by the sleeve tubular body.

19. A surgical aspirator sleeve and tip combination, where the sleeve has an interior surface and the tip has an exterior surface, the combination comprising:
at least one groove formed on at least a portion of the exterior surface of the tip; and
at least one rib formed on the interior surface of the sleeve that is slidably engageable with the grooves for properly aligning the aspirator sleeve with the aspirator tip and restricting longitudinal and rotational movement of said aspirator sleeve relative to the aspirator tip

20. A surgical aspirator sleeve and tip combination, comprising:
a key formed on at least a portion of the surgical aspirator tip; and
a slot formed in at least a portion of the sleeve, wherein the slot slidably receives the key when the aspirator tip is received within the aspirator sleeve to prevent rotational movement of the sleeve with respect to the tip and maintain the alignment of the sleeve with respect to the tip.

21. The combination of claim 20, wherein the key height is at least equal to the thickness of the aspirator sleeve.

22. A surgical aspirator sleeve and tip combination, the aspirator tip having an aspirator tip end and an external surface, and the aspirator sleeve having an internal surface and a distal tip sleeve end portion, the combination comprising:
at least one tip end projection formed on the external surface of the aspirator tip end, the at least one tip end projection extending generally laterally relative to the aspirator tip end; and
at least one sleeve tip end projection formed in the distal tip sleeve end portion, wherein said at least one projection may selectively engage the tip end projection to form a gap between the distal tip sleeve end portion and the aspirator tip end portion, such that liquid or passing through the sleeve can flow freely towards the aspirator tip end.

23. A surgical aspirator sleeve and tip combination, the aspirator tip having an external surface and a tip end portion and the aspirator sleeve having an internal surface and a distal tip sleeve end portion, the combination comprising:
at least one projection formed on the aspirator tip end portion, the projection extending generally laterally relative to the aspirator tip end portion;
at least one pair of converging longitudinal grooved ribs extending along at least a portion of the internal surface of the aspirator sleeve, said pair of longitudinal grooved ribs converging near the forward tip end of the aspirator sleeve, and selectively abutting the at least one projection to form a gap between the aspirator tip end portion and the distal tip sleeve end portion, such that gases, fluid, or materials passing through the sleeve can flow freely towards the aspirator tip end.

24. A method of assembling a sleeve and aspirator tip combination, the method comprising:
(a) providing a surgical aspirator tip, the aspirator tip comprising:
(i) an enlarged medial portion having an internal medial portion surface and an external medial portion surface, said medial portion including at least one longitudinal groove formed on the external medial portion surface;
(ii) a hollow tubular member having an exterior surface, said hollow tubular member extending distally from the medial portion and with the hollow tubular member being diametrically no larger than the enlarged medial portion;
(iii) a tip end portion of the distal end of the hollow tubular neck member opposite the medial portion, said tip end portion defining at least one tip end opening that is in communication with the hollow tubular neck member;
(b) providing a surgical aspirator sleeve, the sleeve comprising:
(i) an elongate, tubular body composed of resilient, deformable material, the body having an internal sleeve surface and an external sleeve surface;
(ii) said tubular body defining an internal channel having an open, proximal aspirator sleeve end portion and an enclosed, distal tip sleeve end portion, said tubular body defining plural, spaced orifices at a spaced distance from the tip end, said orifices providing communication between the external environment and internal channel of the tubular body to allow passage of gases, fluid, or materials to be aspirated into the internal channel defined by the tubular body;
(iii) said tubular body slidably receivable over the hollow tubular neck member of the surgical aspirator tip to assume the profile of the hollow tubular neck member of the aspirator tip;
(iv) wherein the size of the internal channel of said tubular body is sufficiently larger than the exterior of the hollow tubular neck member to enable the gases, fluid, or materials passing into the channel through the orifices to flow through the internal channel between the interior of the tubular body and the exterior of the hollow tubular neck member toward the distal tip end of the hollow tubular neck member;
(c) forming at least one venting channel in communication with the interior of the sleeve and the external environment between the at least one longitudinal exterior groove of the enlarged medial portion and the internal surface of the proximal aspirator sleeve end, allowing airflow between the external environment and the interior of the sleeve;
(d) providing locking means to help secure said tubular body to the aspirator tip and to restrict longitudinal and rotational movement of said tubular body relative to the aspirator tip; and
(e) providing alignment means for guiding the sleeve onto the tip to properly align the tubular body and aspirator tip, thereby helping to ensure the formation of at least one venting channel therebetween.

25. The method of claim 24, further comprising:
(a) providing at least two tip end projections on the tip end portion that extend generally laterally to the hollow tubular neck member, wherein the tip end projections form at least one tip end groove therebetween;
(b) providing at least two sleeve tip end projections formed on the interior surface of the sleeve at the sleeve distal tip end portion, said projections being configured to selectively engage the tip end grooves;
(c) mating the aspirator sleeve with the aspirator tip such that the sleeve tip end projections may abut the tip end projections.

26. The method of claim 24, wherein the open, proximal end portion of the tubular body of the aspirator sleeve is resiliently expandable.

27. The method of claim 24, wherein the internal channel of the enlarged medial portion of the aspirator tip is in communication with the interior of the hollow tubular neck member allowing gases, fluid, or materials to flow from the interior of the hollow neck member through the internal channel of the enlarged medial portion.

28. The method of claim 24, wherein the external sleeve surface has a plurality of outwardly projecting ribs.

29. The method of claim 24, wherein the locking means further comprises providing at least one groove formed on the exterior surface of the enlarged medial portion that slidably engages at least one rib formed on the interior surface of the tubular body.

30. The method of claim 24, wherein the locking means further comprises a key and slot joint.

31. The method of claim 30, wherein the key and slot joint further comprises:
(a) forming a key that protrudes from at least a portion of the external medial portion surface;
(b) forming a slot in the proximal aspirator sleeve end portion, the slot generally conforming to the shape of the key; and
(c) engaging the key with the slot to restrain rotational movement of said sleeve tubular body relative to the aspirator tube.

32. The method of claim 24, wherein the alignment means further comprises providing a key and slot joint.

33. The method of claim 32, wherein the key and slot joint further comprises:
(a) forming a key that protrudes from at least a portion of the external medial portion surface;
(b) forming a slot in the proximal aspirator sleeve end portion, the slot generally conforming to the shape of the key; and
(c) engaging the key with the slot to restrain rotational movement of said sleeve tubular body relative to the aspirator tube.

34. The method of claim 24, wherein the alignment means further comprises providing at least one groove formed on the exterior surface of the enlarged medial portion that slidably engages at least one rib formed on the interior surface of the tubular body.

35. The method of claim 24, wherein the alignment means further comprises providing at least a first indicia formed on a portion of the sleeve external surface and at least a second indicia formed on a portion of the enlarged medial portion, wherein the first indicia may be visually aligned with the second indicia when the tip is being slidably received by the sleeve tubular body.

36. A surgical aspirator tip, comprising:
(a) an enlarged medial portion having an internal channel;
(b) a hollow tubular neck member extending distally from the medial portion and with the hollow tubular neck member being diametrically no larger than the enlarged medial portion; and
(c) a tip end portion at the distal end of the hollow tubular neck member opposite the medial portion, said tip end portion
(i) defining at least one tip end opening that is in communication with the hollow tubular neck member, and
(ii) comprising at least two tip end projections formed on the tip end portion and extending generally laterally to the hollow tubular neck member, wherein the tip end projections form at least one tip end groove therebetween.

37. The surgical aspirator tip of claim 36, the tip end portion further comprising at least one tip end aperture formed in the tip end grooves, said at least one tip end aperture in communication with the tip end opening.

38. The surgical aspirator tip of claim 37, wherein the tip end projections are sized and positioned to enable fluids, gases and materials to flow within the tip end grooves and into the tip end apertures.

39. The surgical aspirator tip of claim 37, wherein the tip end apertures extend generally transversely to the tip end portion, the tip end apertures extending between adjacent tip end grooves and intersecting the tip end opening.

40. The surgical aspirator tip of claim 37, wherein the tip end apertures extend radially outwardly from the tip end opening through the tip end portion to the tip end grooves.

41. The surgical aspirator tip of claim 37, comprising a plurality of tip end apertures located at varying distances from the tip end opening.
